(19) United States
(12) Patent Application Publication
(10) Pub. No.: US 2025/0352633 A1
(43) Pub. Date: Nov. 20, 2025

(54) COMPOSITIONS, METHODS AND USES FOR THERMALLY STABLE BROAD-SPECTRUM HUMAN PAPILLOMAVIRUS FORMULATIONS

(71) Applicants: THE REGENTS OF THE UNIVERSITY OF COLORADO, A BODY CORPORATE, Denver, CO (US); THE JOHNS HOPKINS UNIVERSITY, Baltimore, MD (US); MEDICAL UNIVERSITY OF VIENNA, Vienna (AT)

(72) Inventors: Robert L. Garcea, Boulder, CO (US); Theodore Randolph, Niwot, CO (US); Richard Roden, Severna Park, MD (US); Reinhard Kirnbauer, Sooss (AT)

(21) Appl. No.: 19/069,038

(22) Filed: Mar. 3, 2025

Related U.S. Application Data
(63) Continuation of application No. 17/210,252, filed on Mar. 23, 2021, now abandoned.
Application No. 17/210,252 is a continuation of application No. PCT/US19/53674, filed on Sep. 27, 2019.
(60) Provisional application No. 62/738,705, filed on Sep. 28, 2018.

(51) Int. Cl. A61K 39/12; A61K 39/00; C12N 7/00

(52) U.S. Cl. CPC A61K 39/12; C12N 7/00; A61K 2039/5258; A61K 2039/55505; C12N 2710/20034

(57) ABSTRACT

Embodiments of the present invention provide for novel compositions and methods for making and using a thermally stable broad spectrum human papilloma virus (HPV) vaccine or immunogenic formulation. Certain embodiments concern lyophilizing HPV formulations in the presence or absence of adjuvants. Other embodiments concern lyophilizing HPV capsomere vaccines and other immunogenic agents to increase stability or reduce degradation of HPV peptides to prolong storage, delivery and use. In yet other embodiments, a single immunogenic formulation can include a thermally stable composition of a broad-spectrum HPV immunogenic composition against multiple HPV types. In some embodiments, a stabilizing formulation can include RG1 HPV16VLP antigens in a hypertonic mixture of a disaccharide and a volatile buffer.

### RELATED APPLICATIONS

This application is a continuation of U.S. patent application Ser. No. 17/210,252, filed Mar. 23, 2021, which is a continuation of PCT Application No. PCT/US19/53674 filed Sep. 27, 2019, which claims priority to U.S. Provisional Application No. 62/738,705, filed Sep. 28, 2018. These applications are incorporated herein by reference in their entireties for all purposes.

### FEDERAL FUNDING

This invention was made with government support under grant number P50 CA 098252-12 awarded by the National Institutes of Health. The government has certain rights in the invention.

### FIELD

Embodiments of the present invention provide for novel compositions and methods for making and using a thermally stable broad-spectrum human papilloma virus (HPV) vaccine or immunogenic formulation. Certain embodiments concern lyophilizing HPV complexes in the presence of various agents to increase stability or reduce degradation of HPV peptides prolonging storage stability, delivery and use. In yet other embodiments, a single immunogenic formulation can include a thermally stable composition of a broad-spectrum HPV immunogenic composition against multiple HPV types. In some embodiments, a stabilizing formulation can include a hypertonic mixture including one or more disaccharide and one or more volatile salts for lyophilization and prolonged storage of RG1-VLP antigens (e.g. RG1 HPV16VLP) or the like. In yet another embodiment, exposure to increased temperatures of a stabilized, lyophilized broad-spectrum HPV complex (e.g. RG1-VLP) re-constituted construct can increase cross-reactivity of the complex against multiple HPV types compared to lyophilized HPV complexes not exposed to elevated temperatures.

### BACKGROUND

Papillomaviruses can infect a wide variety of different species including humans and other mammals. Infection can lead to benign epithelial and fibro-epithelial tumors, or warts at the site of infection. Species-specific sets of papillomaviruses infect a particular species, including several different papillomavirus types. More than one hundred different human papillomavirus (HPV) genotypes have been isolated. For example, canine and rabbit papillomaviruses cannot induce papillomas in heterologous species such as humans. Neutralizing immunity to infection against one papillomavirus type typically is unable to confer immunity against another type, even when the types infect a homologous species.

In humans, papillomaviruses can cause genital warts, which is a prevalent sexually-transmitted condition. HPV low risk (lr) types 6 and 11 are most commonly associated with benign genital warts (e.g., condylomata acuminate). While most HPV-induced lesions are benign, lesions arising from certain high-risk (hr) papillomavirus types e.g., HPV-16 and HPV-18, can undergo malignant progression. Moreover, infection by one of the malignancy-associated papillomavirus types is considered to be a significant risk factor in the development of cervical cancer. Cervical cancer is the third most common cancer in women worldwide. Most cervical cancer cases occur in women living in developing countries where availability of vaccines and preventative screenings, such as pap smears are limited.

Delivering an effective HPV vaccine to developing countries comes with many challenges. For example, cost of an HPV vaccine for developing countries needs to be relatively inexpensive. Further, keeping vaccines at a temperature sufficient to maintain the composition and reduce degradation can be difficult when delivering vaccines to remote regions and limited refrigerated space is available for vaccine storage. The recommended temperature ranges for transporting vaccines in refrigeration or cooler temperatures are narrow and technology for maintaining temperatures of vaccines within these ranges can be unavailable if delivering vaccines to a developing country. Therefore, one of the issues involved with the production and use of HPV vaccines has been providing effective storage and transportation of the vaccines where storage conditions can reduce degradation or increase stability of a viral vaccine formulation.

### SUMMARY

Embodiments of the present invention provide for novel compositions and methods for a thermally stable broad-spectrum human papilloma virus (HPV) formulation. Certain aspects disclosed herein concern partially or fully lyophilizing or freeze-drying the broad-spectrum HPV vaccine formulation in the presence of a hypertonic mixture. Other embodiments described herein concern freeze-drying broad-spectrum HPV constructs (e.g., RG1 HPV16VLPs) to increase stability or decrease degradation or disassembly of the constructs during storage, transportation, delivery resulting in a reduction of product loss and reduction of loss of efficacy.

In some embodiments, broad spectrum HPV VLPs can be lyophilized and dried to create powdered formulations. In certain embodiments, constructs can include RG1 HPV16VLPs or similar construct representing multiple HPV serotypes (U.S. Pat. No. 9,149,503 is incorporated herein in its entirety for all purposes).

In certain embodiments, one approach to increase the type spectrum response for increasing protection and reducing the risk of HPV infection can be based upon L2 minor capsid protein. The L2 N-terminus of papillomaviruses contains type-common epitopes and immunizations with L2 proteins or peptides induce low titers of cross-neutralizing antibodies. In some embodiments, a twenty amino acid polypeptide fragment (e.g. aa17-36) ‘RG1’ of HPV16 L2 has been demonstrated as a broad cross-neutralization epitope. For example, RG1-VLP are empty capsids self-assembled from a chimeric HPV16L1-16RG1 fusion protein, that repetitively (about 360×) present RG1 on the HPV16 L1-VLP surface via a DE surface-loop. RG1-VLP immunizations induce robust HPV16-neutralizing antisera and provide (cross-) protection against all known 13 mucosal hr types, several mucosal Ir and even distantly related cutaneous HPV have demonstrated some protection or reduced risk. Certain embodiments disclosed herein concern RG1-VLPs of use in formulations against two or more HPV serotypes.

In other embodiments, compositions disclosed herein include, but are not limited to, one or more volatile salts. In accordance with these embodiments, one or more volatile salts can include, but are not limited to, one or more of ammonium acetate, ammonium formate, ammonium carbonate, ammonium bicarbonate, tricthylammonium acetate, triethylammonium formate, triethylammonium carbonate, trimethylamine acetate trimethylamine formate, trimethylamine carbonate, pyridinal acetate and pyridinal formate, or combinations thereof.

In other embodiments, formulations of use herein can include one or more non-reducing disaccharides including, but not limited to, trehalose, sucrose and lactose, and additional glass forming agents. Glass-forming agents can include, but are not limited to, hydroxyethyl starch, glycine, glycine and mannitol, cyclodextrin, and polyvinyl pyrrolidone (povidone) or combinations thereof.

In some embodiments, formulations of use herein can include a VLP assembled from an HPV L1 protein, one or more disaccharide and one or more volatile salt or volatile salt buffer. In accordance with these embodiments, a VLP assembled from an HPV L1 protein can be from HPV16, a disscharide can include one or more of trehalose, sucrose, lactose, maltose or the like and one or more volatile salts can include one or more of ammonium acetate, ammonium formate, ammonium carbonate, ammonium bicarbonate, triethylammonium acetate, or the like. In certain embodiments, a stabilizing formulation of use to prolong shelf-life of RG1 HPV16VLPs or similar constructs can include a hypertonic mixture including trehalose and ammonium acetate.

In certain aspects of the instant disclosure, immunogenic formulations of broad-spectrum HPV constructs including VLPs can be lyophilized for example, where the broad-spectrum construct remains intact. In addition, these combinations can reduce detrimental modifications to critical neutralization epitopes of an assembled VLP. In other embodiments, broad-spectrum HPV construct compositions disclosed herein preserve immunogenicity e.g. ability to induce neutralizing antibody titer by increasing stability and/or decreasing disassembly or degradation. In other embodiments, antigen compositions described herein can be stabilized to preserve immunogenicity (e.g. reduce antibody titer loss) following incubating lyophilized complexes at temperatures of about 40° C., to about 50° C. to about 60° C., to about 70° C. degrees for a few hours, to a day, to up to several days, up to a week, up to several weeks, up to a month or up to several months making it possible to store and transport these lyophilized compositions at an increased temperature for a longer duration. In certain embodiments, immunogenic compositions of RG1 HPV16VLPs or the like can be frozen on precooled shelves of a lyophilizer and dried under vacuum creating an essentially dry powder formulation. In other embodiments, RG1 HPV16VLPs or similar multi-targeted HPV construct formulations can include particulate adjuvants such as aluminum or aluminum salt adjuvants; for example, aluminum hydroxide but not limited to, one or more of aluminum hydroxide, aluminum phosphate and aluminum sulfate, or combinations thereof. In other embodiments, compositions disclosed herein can include a disaccharide or glass-forming agent such as trehalose as well as a volatile salt such as ammonium acetate. In accordance with these embodiments, multi-targeted HPV construct formulations can be lyophilized (e.g. rapid drying or tray-dried) to prolong shelf life of the active agents.

In yet other embodiments, these stored formulations can be reconstituted for use as an immunogenic formulation against infection with multiple HPV types after exposure to elevated temperatures of about 40 to about 70° C. for a day to months to enhance HPV-type cross reactivity, if desired. In certain embodiments, an L2 component of the RG1 HPV VLP construct cross-reactivity can be enhanced to induce greater cross-reactivity against a broad range of HPV types, at the same time or sequentially. In other embodiments, vaccine formulations described herein can be used alone or in combination with other agents to prevent or reduce the onset of HPV infections in a subject (e.g., GARDASIL™ and CERVARIX™).

In other embodiments, vaccine or immunogenic compositions disclosed herein can contain RG1 HPV16VLPs or similar HPV broad-spectrum multi-antigen constructs. In accordance with these embodiments, immunogenic compositions disclosed herein can also contain particulate adjuvants such as aluminum or aluminum salts, for example aluminum hydroxide or aluminum hydroxide with glycopyranoside lipid A (GLA), as well as disaccharide agents, such as trehalose and/or sucrose. In some embodiments, these immunogenic compositions can be co-lyophilized, stored and/or transported to remote areas where they can be reconstituted with no loss of multimeric structure or immunogenicity.

In yet other embodiments, stored lyophilized formulations disclosed herein can be stored at elevated temperatures (at about 40 to about 70° C.) and subsequently reconstituted for use against the multiple targeted HPV serotypes of low or high risk. In certain embodiments, the broad spectrum multi-targeted antigen complexes can be stored at elevated temperatures (at about 40 to about 60° C.) for a few hours, to one day, to several days, to a week, several weeks, or a month or 2 months or 3 months or more, prior to reconstitution to enhance cross reactivity of the multi-targeted antigen complex against two or more targets (e.g. pathogens or serotypes).

In other embodiments, vaccine or immunogenic compositions disclosed herein can contain broad-spectrum HPV constructs. In accordance with these embodiments, immunogenic compositions disclosed herein can also contain particulate adjuvants such as aluminum or aluminum salt adjuvants, for example aluminum hydroxide or aluminum hydroxide with glycopyranoside lipid A (GLA), as well as disaccharide agents or glass-forming agents, such as trehalose and/or sucrose in combination with broad-spectrum multi-targeted antigen constructs. In some embodiments, these immunogenic compositions can be co-lyophilized, and/or stored at elevated temperatures and/or transported to remote areas where they can be reconstituted with little to essentially no loss of multimeric structure or immunogenicity of the constructs, or change in the adjuvant particle size distribution.

### BRIEF DESCRIPTION OF THE FIGURES

The following drawings form part of the instant specification and are included to further demonstrate certain aspects of particular embodiments disclosed herein. The embodiments may be better understood by reference to one or more of these drawings in combination with the detailed description presented herein.

FIG. 1 represents a table reflecting exemplary data where serum antibody titers in mice raised by vaccination with a construct (lyophilized RG1 HPV16 VLP) of various embodiments disclosed herein and incubated at indicated temperatures and times was measured.

FIGS. 2A and 2B represent some data obtained from an exemplary neutralization assays (L1-PBNA, L2-PBNA) to detect serum neutralization titers against HPV16 as well as some cross-neutralization data against other indicated HPV types raised by immunization of mice with certain embodiments disclosed herein (lyophilized RG1 HPV16 VLP stored at indicated times and temperatures).

FIG. 3 represents a histogram plot of a mouse model testing stimulation of T cell responses after immunization with a broad-spectrum HPV complex stored in exemplary compositions at various indicated temperatures for 1 month, when splenocytes are exposed to various indicated agents including positive and negative controls.

FIG. 4 represents an image of an exemplary negative stain electron micrograph of RG1-HPV16 VLPs demonstrating intact VLPs in certain embodiments disclosed herein.

FIG. 5 represents a table reflecting exemplary data where serum antibody ELISA titers in mice raised by vaccination with a construct (RG1 HPV16 VLP) of various embodiments disclosed herein and incubated at indicated temperatures for 1 month were measured illustrating effects of lyophilized compared to liquid vaccine formulations.

FIGS. 6A and 6B are tables of representative data illustrating effects of indicated temperatures on lyophilized compared to liquid formulations of RG1-HPV16 VLP with respect to induction of neutralizing antibody titers to indicated HPV types in mice following vaccination with various broad-spectrum multi-targeted complexes of certain embodiments disclosed herein.

FIG. 7 is a histogram plot of IFN-γ induction by ELISPOT in mice based on T cell response to indicated stimuli following vaccination with RG1 HPV16 VLP antigen stored under indicated temperature conditions for 1 month comparing lyophilized to liquid formulations.

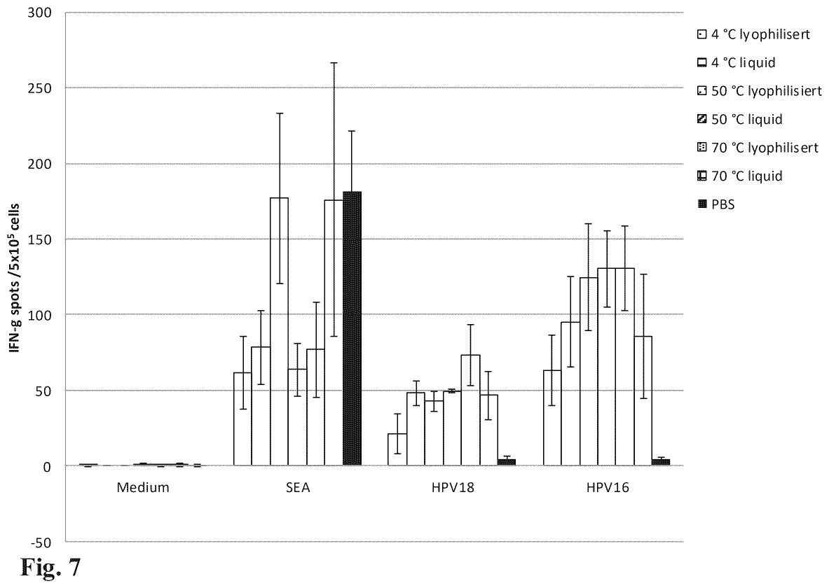

### DEFINITIONS

As used herein, “a” or “an” may mean one or more than one of an item.

As used herein, “about” may mean up to and including plus or minus five percent, for example, about 100 may mean 95 and up to 105.

Capsid protein: the structural protein of a virus, e.g., enveloped or non-enveloped, which constitutes the capsid structure. Generally, there are several capsid proteins which are often described by whether they are the predominant (major) constituent or lesser (minor) constituent of capsid structure.

Conformational antibody: an antibody that specifically binds an epitope expressed as a correctly-folded L1 or L2 protein but not on denatured L1 or L2 protein.

Capsomere: this refers to a structure that makes up the larger viral capsid structure that is generally a pentamer of one type of capsid proteins. In the case of HPV, a native capsomere comprises a pentamer of L1 capsid proteins that may be associated with one L2 capsid protein.

“Capsid” as used herein refers to the structural portion of a virus, e.g., HPV that is comprised of capsomeres. In the case of HPV, the viral capsid is comprised of 72 capsomeres.

### DETAILED DESCRIPTIONS

In the following sections, various exemplary compositions and methods are described to detail various embodiments. It will be obvious to one skilled in the art that practicing the various embodiments does not require the employment of all or even some of the details outlined herein, but rather that concentrations, times and other details may be modified through routine experimentation. In some cases, well known methods or components have not been included in the description.

In certain embodiments, compositions, methods and uses for stabilizing HPV vaccine formulations are disclosed. A formulation or application of a formulation that can stabilize viral vaccines from for example, from degradation or disassembly of a viral structure is disclosed. In certain embodiments, compositions disclosed herein can be used to reduce loss of titer of lyophilized HPV formulations. In certain embodiments, compositions disclosed herein can concern a combination of two or more agents (e.g., adjuvant or adjuvant-like agent) provided to an HPV vaccine formulation where the formulation is then lyophilized.

In some embodiments, vaccine formulations can be lyophilized in the presence of one or more disaccharide and one or more volatile salt and sufficient liquid can be removed during lyophilization that the dried or essentially dried vaccine formulation or immunogenic composition is stabilized from degradation. For example, the anticipated storage temperature may be room temperature or higher.

Embodiments of the present invention provide for novel compositions and methods for a thermally stable broad-spectrum human papilloma virus (HPV) formulation. Certain aspects concern partially or fully lyophilizing or freeze-drying the broad-spectrum HPV formulation in the presence of a hypertonic mixture. Other embodiments described herein concern freeze-drying broad-spectrum HPV constructs (e.g. RG1 HPV16VLPs) to increase stability or decrease degradation or disassembly of the constructs during storage, transportation, delivery resulting in a reduction of product loss and reduction of loss of efficacy.

In some embodiments, broad spectrum HPV VLPs are lyophilized and dried to create powdered formulations. In certain embodiments, constructs can include RG1-VLPs, RG1 HPV16VLPs or similar (U.S. Pat. No. 9,149,503 is incorporated herein in its entirety for all purposes).

In certain embodiments, compositions disclosed herein include, but are not limited to, one or more volatile salts. In accordance with these embodiments, one or more volatile salts can include, but are not limited to, one or more of ammonium acetate, ammonium formate, ammonium carbonate, ammonium bicarbonate, triethylammonium acetate, tricthylammonium formate, tricthylammonium carbonate, trimethylamine acetate trimethylamine formate, trimethylamine carbonate, pyridinal acetate and pyridinal formate, or combinations thereof.

In other embodiments, formulations of use herein can include one or more non-reducing disaccharides including, but not limited to, trehalose, sucrose and lactose, and additional glass-forming agents, as necessary, including, but not limited to, hydroxyethyl starch, glycine, glycine and mannitol, cyclodextrin, and polyvinyl pyrrolidone (povidone) or combinations thereof.

It is known that state-of-the art HPV vaccines confer protection against a limited number of high-risk (hr) and low-risk (lr) HPV types (e.g. Cervarix™, Gardasil™ and Gardasil-9® target 2 or 7 of 13 hr types, the latter also 2 Ir types). These commercially available compositions are based upon the major structural protein L1 assembled into highly immunogenic virus-like particles (VLP) that induce high titers of type-specific neutralizing antibodies. Further, these HPV vaccines are expensive in part due to their complex multivalent formulation and dependency on cold storage, which makes vaccine distribution difficult regarding these state-of-the-art formulations particularly for poorer countries that carry the majority of cervical cancer burden and have reduced resources.

In some embodiments, formulations of use herein can include a VLP construct assembled from an HPV L1 protein, one or more disaccharide and one or more volatile salt and/or volatile salt buffer. In accordance with these embodiments, a VLP assembled from an HPV L1 protein can be from HPV16, a disaccharide can include one or more of trehalose, sucrose, lactose, maltose or the like and one or more volatile salts can include one or more of ammonium acetate, ammonium formate, ammonium carbonate, ammonium bicarbonate, triethylammonium acetate, or the like. In accordance with these embodiments, a stabilizing formulation of use to prolong shelf-life of an exemplary VLP construct can include RG1-VLPs (where for example, an alternative peptide derived from a pathogenic agent can be inserted in addition to an HPV L1 such as HPV16 L1) or similar constructs combined in a hypertonic mixture. In certain embodiments, the hypertonic mixture can include trehalose and ammonium acetate.

In certain aspects of the instant disclosure, immunogenic formulations of broad-spectrum HPV immunogenic formulations including VLPs can be lyophilized for example, where the broad-spectrum construct remains intact. In addition, these combinations can reduce detrimental modifications to critical neutralizing epitopes of an assembled HPV L1 VLP complex. In other embodiments, broad-spectrum HPV construct compositions disclosed herein preserve antibody titer by increasing stability and/or decreasing disassembly or degradation. In certain embodiments, antigen compositions described herein can be stabilized in order to reduce antibody titer loss at temperatures of about 40° C. to about 50° C. to about 60° C. degrees for up to several weeks to several months making it possible to store and transport these compositions at an increased temperature for a longer duration. In certain embodiments, immunogenic compositions of RG1-VLPs or the like can be frozen on precooled shelves of a lyophilizer and dried under vacuum creating an essentially dry powder formulation. In other embodiments, RG1 HPV16VLPs or the like formulations including trehalose and ammonium acetate can be lyophilized and dried to prolong shelf-life of the active agents for storage and transport.

In other embodiments, these stored formulations can be reconstituted for use against multiple HPV types. In other embodiments, vaccine formulations described herein can be used alone or in combination with other agents used to prevent HPV infections in a subject (e.g., GARDASIL™ and CERVARIX™).

In some embodiments, an HPV protein complex as part of an immunogenic composition disclosed herein can include VLPs of HPV L 1 having an L2 minor capsid peptide substitution. In other embodiments, vaccine or immunogenic compositions disclosed herein can contain RG1 HPV16VLPs or similar HPV broad-spectrum constructs. In accordance with these embodiments, immunogenic compositions disclosed herein can also contain particulate adjuvants such as aluminum or aluminum salt adjuvants, for example aluminum hydroxide or aluminum hydroxide with glycopyranoside lipid A (GLA), as well as disaccharides, such as trehalose and/or sucrose. In some embodiments, these immunogenic compositions can be co-lyophilized, stored and/or transported to remote areas where they can be reconstituted with no loss of multimeric structure or immunogenicity.

### RG1-VLPs

One recent approach to increase the spectrum of protection to broader HPV types is based upon an L2 minor capsid protein. The L2 N-terminus of papillomaviruses contains type-common epitopes and immunizations with L2 proteins or peptides induce low titers of cross-neutralizing antibody. In certain embodiments, a twenty amino acid (e.g. aa 17-36) peptide ‘RG1’ of HPV16 L2 has been described as a broadly cross-neutralization epitope and is contemplated of use herein.

As previously disclosed, RG1-VLPs exist as empty capsids self-assembled from a chimeric HPV16L1-16RG1 fusion protein, that repetitively (e.g. 360×) presents RG1 on the HPV16 L1-VLP surface through a DE surface-loop. It has been demonstrated that RG1-VLP immunizations induce robust HPV16-neutralizing antisera and provide (cross-) protection against all 13 mucosal high-risk types, several mucosal low-risk types and even distantly related cutaneous HPV. RG1-VLP have been produced and are available.

In some embodiments disclosed herein, complex HPV multi-antigen-containing immunogenic constructs can be used in compositions and co-lyophilized in the presence of various agents (e.g. aluminum hydroxide or similar particulate adjuvant), stored in elevated temperatures and/or transported to remote areas where they can be reconstituted with little to no loss of multimeric structure or immunogenicity.

In certain embodiments, a buffer of use in compositions disclosed herein can include, but is not limited to, one or more volatile salts. In accordance with these embodiments, one or more volatile salts can include, but are not limited to, one or more of ammonium acetate, ammonium formate, ammonium carbonate, ammonium bicarbonate, tricthylammonium acetate, tricthylammonium formate, tricthylammonium carbonate, trimethylamine acetate trimethylamine formate, trimethylamine carbonate, pyridinal acetate and pyridinal formate, or combinations thereof.

In other embodiments, a non-reducing disaccharide disclosed herein can include one or more of trehalose, sucrose, lactose, or combinations thereof. In some embodiments, the disaccharide concentration in a weight-to-volume (w/v) can be from about 1% to about 20%, or about 5% to about 15% (w/v) in a liquid vaccine formulation prior to freeze drying. In other embodiments, the glass-forming agent can be trehalose present in a concentration of from about 1% to about 20% w/v or about 5% to about 15% w/v or about 8% to about 20% w/v in the liquid vaccine formulation prior to freeze drying/lyophilizing. In another embodiment, the glass-forming agent can be trehalose at a concentration of about 10% w/v in the liquid vaccine formulation or immunogenic composition prior to freeze-drying.

In some embodiments, compositions disclosed herein can include a hypertonic buffer composed of volatile salts and a disaccharide agent at various concentrations (1.0% to 20% (w/v)) prior to lyophilization. In certain embodiments, concentrations of these agents can be from about 1% to about 30% w/v. In other embodiments, a broad-spectrum multi-antigen HPV construct disclosed herein can be included in a stabilizing composition for lyophilization or other purpose can be from about 0.01 mg/mL to about 5.0 mg/mL, or about 0.01 mg/mL to about 3.0 mg/mL; or about 0.01 mg/mL to about 2.0 mg/mL; or about 0.05 mg/mL to about 1.5 mg/mL. In some embodiments, a broad-spectrum HPV construct disclosed herein included in a stabilizing composition for lyophilization or other purpose can be from about 0.05 mg/mL to about 2.0 mg/mL.

In some embodiments, stability of vaccine or immunogenic compositions disclosed herein can be enhanced by the addition of nonionic surfactants. In accordance with these embodiments, surfactants can be added to vaccine or immunogenic formulations at concentrations ranging from approximately 0.1 times the critical micelle concentration of the surfactant in the vaccine composition, to approximately 20 times the critical micelle concentration of the surfactant in the vaccine composition before, during or after lyophilization of the composition. Suitable nonionic surfactants include, but are not limited to, polsorbates such as Tween 20, Tween 40, Tween 60 and Tween 80, polaxamers for example Polaxamer 188 and Polaxamer 407, Poloxamer 235, Poloxamer 335, Brij, alkylphenol hydroxypolyethylene surfactants such as Triton X100, Triton X114 and Triton X405, and Oligoethylene glycol monoalkyl ethers such as Genapol.

In some embodiments, the aluminum salt adjuvant of the vaccine composition can include one or more of aluminum hydroxide, aluminum phosphate and aluminum sulfate, or combinations thereof. In other embodiments, the aluminum salt can be in the form of an aluminum hydroxide gel (e.g., ALHYDROGEL™) or other consistency. In certain embodiments, the aluminum salt adjuvant includes aluminum hydroxide. In some embodiments, a broad-spectrum multi-antigen HPV construct (e.g. RG1-VLPs) can be combined with an aluminum salt adjuvant, for example, aluminum hydroxide (aluminum agent: ‘alum’) at a ratio of 1 μg complex to 5 μg aluminum salt adjuvant. Other ratios contemplated herein can be 1:1; 1:2; 1:3; 1:4; 1:6; 1:7; 1:10; 1:15; 1:20 or the like.

In some embodiments, thermal stability of tertiary structure of a broad-spectrum HPV complex can be assessed by any method known in the art. In other embodiments, thermal stability of tertiary structure of a broad-spectrum HPV complex can be assessed using various methods including, but not limited to, front-face fluorescence. For example, front-face fluorescence can be used to examine tertiary structures of RG-1 HPV16 L1 capsomeres. In certain embodiments, front face fluorescence can use acrylamide quenching to assess the tryptophan environment in each vaccine formulation, and a Stern-Volmer constant can be calculated based on the fluorescence. A high Stern-Volmer constant is generally indicative of greater tertiary instability, which allows tryptophan residues to be more easily quenched. For example, a lower Stern-Volmer constant is generally indicative of less tertiary instability (i.e., a more native protein structure), which reduces tryptophan quenching. In certain embodiments, these comparisons can be made on a complex to assess stability of the complex at a given temperature in compositions described herein.

In some embodiments, immunogenic compositions disclosed herein can further include a co-stimulatory agent. In accordance with these embodiments, a co-stimulatory agent may be added to a composition prior to lyophilization of a formulation. Co-stimulatory agents contemplated of use herein can include, but are not limited to, one or more of lipid A, lipid A derivatives, monophosphoryl lipid A, chemical analogues of monophosphoryl Lipid A, CpG containing oligonucleotides, TLR-4 agonists, flagellin, flagellins derived from gram negative bacteria, TLR-5 agonists, fragments of flagellins capable of binding to TLR-5 receptors, saponins, analogues of saponins, QS-21, purified saponin fractions, ISCOMS and saponin combinations with sterols and lipids, or combinations thereof. In other embodiments, the co-stimulatory agent can be about 0.05 mg/mL Glycopyranoside lipid A (GLA) or similar agent having similar effects.

### VLPs and Capsomeres

Virus-like particles or VLPs: the capsid-like structures that result upon expression and assembly of a papillomavirus L1 DNA sequence alone or in combination with an L2 DNA sequence. VLPs are morphologically and antigenically similar to authentic virions. VLPs may be produced in vivo, in suitable host cells or may form spontaneously upon purification of recombinant L1 and/or L2 proteins. Alternatively, they may be produced using capsid proteins L1 and L2, fragments or mutated forms thereof, e.g., L1 or L2 proteins that have been modified by the addition, substitution or deletion of one or more amino acids. L1 and L2 mutants that fall within the scope of the present invention are those that upon expression present at least one native PV conformational epitope. Methods to assemble VLPs are known in the art, as would be readily appreciated and is understood by one of ordinary skilled based on the present disclosure.

Correctly-folded L1 or L2 protein: L1 or L2 protein, fragment thereof, or mutated form thereof, (either monomeric, in the form of small oligomers (dimers-tetramers) or (capsomeres), which, upon expression, assumes a conformational structure that presents one or more conformational HPV L1 or L2 epitopes present on native viral capsids or VLPs and is suitable for assembly into VLPs. In the present invention, a correctly folded HPV L1 or L2 protein will present one or more HPV L1 or L2 conformational epitopes.

A conformational L1 or L2 HPV epitope: generally, refers to an epitope expressed on the surface of correctly-folded L1 or L2 protein which is also expressed by an L1 or L2 protein or fragment, or mutated form thereof, which is also expressed by an L1 or L2 protein of a corresponding wild-type, infectious HPV. It is well accepted by those skilled in the art that the presentation of conformational epitopes is essential to the efficacy (both as prophylactic and diagnostic agents) of HPV L1 or L2 protein immunogens.

A conformational neutralizing L1 or L2 HPV epitope: generally, refers to an epitope expressed on the surface of correctly-folded L1 protein, fragment or mutated form thereof, which is also expressed by an L1 or L2 protein of a corresponding wild-type, infectious HPV, and which elicits neutralizing antibodies. It is well accepted by those skilled in the art that the presentation of conformational neutralizing epitopes is essential to the efficacy (both as prophylactic and diagnostic agents) of HPV L1 or L2 protein immunogens.

Immunogenic epitopes are those that confer protective immunity, allowing a mammal or other animal to resist (delayed onset of symptoms or reduced severity of symptoms), as the result of its exposure to the antigen of a pathogen, disease or death that otherwise follows contact with the pathogen. Protective immunity can be achieved by one or more of the following mechanisms: mucosal, humoral, or cellular immunity. Mucosal immunity is primarily the result of secretory IgA (sIGA) antibodies on mucosal surfaces of the respiratory, gastrointestinal, and genitourinary tracts. The sIGA antibodies are generated after a series of events mediated by antigen-processing cells, B and T lymphocytes that result in sIGA production by B lymphocytes on mucosa-lined tissues of the body. “Humoral immunity” is the result of IgG antibodies and IgM antibodies in serum. “Cellular immunity” can be achieved through cytotoxic T lymphocytes or through delayed-type hypersensitivity that involves macrophages and T lymphocytes, as well as other mechanisms involving T cells without a requirement for antibodies. The primary result of protective immunity is the destruction of the pathogen or inhibition of its ability to replicate itself.

In some embodiments, constructs disclosed herein can be assembled into a VLP. In this embodiment, assembly can be performed using methods known in the art. The present invention includes methods to assemble a VLP using capsomeres of the present invention at acidic to physiological pH. Most preferred are methods to assemble VLPs using capsomeres of the present invention at physiologic pH. In the case of polypeptide sequences that are less than 100% identical to a reference sequence, the non-identical positions are preferably, but not necessarily, conservative substitutions for a particular targeted sequence.

Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. Similar minor variations may also include amino acid deletions or insertions, or both. Guidance in determining which amino acid residues may be substituted, inserted, or deleted without abolishing biological or immunological activity may be found using computer programs well known in the art.

In some embodiments, the HPV L1 or L2 DNA disclosed herein are derived from an HPV which is involved in cancer or condylomata acuminata, e.g., HPV-16, HPV-18, HPV-31, HPV-33, HPV-35, HPV-39, HPV-45, HPV-51, HPV-52, and HPV-56 are involved in cancer, and HPV-6, HPV-11, HPV-30, HPV-42, HPV-43, HPV44, HPV-54, HPV-55, and HPV-70, are involved in warts. However, the subject capsid proteins may be produced using any HPV L1 DNA and further include L2 DNA, depending on the desired response.

Proteins and capsomeres disclosed herein can be produced in a variety of ways, including production and/or recovery of natural proteins, production and/or recovery of recombinant proteins, and/or chemical synthesis of the proteins. The proteins and polypeptides disclosed herein can be expressed in a prokaryotic microbial host, e.g., bacteria such as E. coli that can be cultured under conditions that favor the production of capsid proteins. This will largely depend upon the selected host system and regulatory sequences contained in the vector, e.g., whether expression of the capsid protein requires induction. Proteins and polypeptides of the present disclosure may also be expressed in any host cell that provides for the expression of recoverable yields of the polypeptides in appropriate conformation. Suitable host systems for expression of recombinant proteins are well known and include, by way of example, bacteria, mammalian cells, yeast, and insect cells. One expression system of use to produce complexes disclosed herein can include E. coli expression system used in the Examples, as this system provides for high capsomere yields. However, HPV L1 and L2 proteins, as well as other viral capsid proteins, can be produced in other systems. For example, yeast and baculovirus-infected insect cell cultures can be used.

Suitable vectors for cloning and expressing polypeptides of the present invention are well known in the art and commercially available. Further, suitable regulatory sequences for achieving cloning and expression, e.g., promoters, polyadenylation sequences, enhancers and selectable markers are also well known. The selection of appropriate sequences for obtaining recoverable protein yields is routine to one skilled in the art.

Other embodiments can include polynucleotides that encode chimeric proteins and complexes/capsomeres. Accordingly, any nucleic acid sequence, which encodes the amino acid sequence of chimeric proteins and complexes/capsomeres, can be used to generate recombinant molecules that express chimeric proteins and complexes/capsomeres. It will be appreciated by those skilled in the art based on the present disclosure that as a result of the degeneracy of the genetic code, a multitude of nucleotide sequences encoding chimeric proteins and complexes/capsomeres of the present disclosure, some bearing minimal homology to the nucleotide sequences of any known and naturally occurring gene, may be, produced. Therefore, the disclosure contemplates each and every possible variation of nucleotide sequence that could be made by selecting combinations based on possible codon choices. These combinations are made in accordance with the standard triplet genetic code as applied to the nucleotide sequence of naturally occurring chimeric proteins and complexes/capsomeres of the present disclosure, and all such variations are to be considered as being disclosed.

Certain embodiments of the present application include polypeptides that elicit an immune response to two or more HPV antigens in a subject. An elicited immune response may be either prophylactic, preventing later infection by the specific viral type targeted, or may be therapeutic, reducing the severity of disease. An immune response includes a humoral, e.g., antibody, response to that antigen and/or a cell-mediated response to that antigen. Methods to measure an immune response are known to those skilled in the art. If one or both types of immune response are present, they may protect a subject from any disease caused by an agent, for example, by the agent from which the viral complex was derived. In accordance with the present disclosure, the ability of an immunogenic composition to protect or treat a subject in need thereof from disease can refer to the ability of a capsomere or chimeric protein of the present disclosure to treat, ameliorate and/or prevent disease or infection caused by the agent or cross reactive agent, by eliciting an immune response against an antigen derived from the disease-causing agent and contained within a protein or capsomere of the present disclosure. It is to be noted that a subject may be protected by an immunogenic composition disclosed herein even without detection of a humoral or cell-mediated response to the immunogenic composition. Protection or reducing the risk of developing a viral infection can be measured by methods known to those skilled in the art.

In certain aspects, because it is known that more than one HPV type may be associated with an HPV infection, vaccines or immunogenic compositions can include stable HPV capsid proteins derived from more than one type of HPV where the compositions have been lyophilized with glass-forming excipients and/or aluminum adjuvants to increase their stability to non-refrigerated temperatures. For example, HPV 16 and 18 are known to be associated with cervical carcinomas; therefore, a vaccine for cervical neoplasia can include VLPs of HPV 16; of HPV 18; or both HPV 16 and 18. In fact, a variety of neoplasias are known to be associated with PV infections. For example, HPVs 3a and 10 have been associated with flat warts. A number of HPV types have been reported to be associated with epidermodysplasia verruciformis (EV) including HPVs 3a, 5, 8, 9, 10, and 12. HPVs 1, 2, 4, and 7 have been reported to be associated with cutaneous warts and HPVs 6b, 11a, 13, and 16 are associated with lesions of the mucus membranes. In accordance with these embodiments, a subject vaccine formulation can include a mixture of RG1-VLP constructs.

Other embodiments concern pharmaceutical immunogenic compositions for use in reducing the risk of onset or treating a condition caused by a pathogenic virus or more than one pathogenic virus (e.g., HPV; HPV serotypes). Any known pharmaceutically acceptable excipient is contemplated herein.

Yet another aspect of the present disclosure is a method to elicit an immune response to a chimeric protein or capsomere of a lyophilized or dehydrated composition (after hydration), comprising administering to the subject a composition disclosed herein. The vaccines will be administered in prophylactically or therapeutically effective amounts. That is, in amounts sufficient to produce a protective immunological response. Generally, the vaccines will be administered in dosages ranging from about 0.1 mg protein to about 20 mg protein, more generally about 0.001 mg to about 1 mg protein. Single or multiple dosages can be administered.

Administration of a capsid protein-containing vaccines may be affected by any pharmaceutically acceptable means, e.g., parenterally, locally or systemically, including by way of example, oral, topical, intranasal, intravenous, intramuscular, and topical administration. The manner of administration is affected by factors including the natural route of infection. The dosage administered will depend upon factors including the age, health, weight, kind of concurrent treatment, if any, and nature and type of the virus, e.g., human, papillomavirus. The vaccine may be employed in dosage form such as capsules, liquid solutions, suspensions, or elixirs, for oral administration, or sterile liquid formulations such as solutions or suspensions for parenteral or intranasal use.

In yet other embodiments, multi-targeted HPV antigen complexes can be lyophilized and stored in elevated temperatures of about 40° C. to about 60° C. for a pre-determined period of days to months (e.g. 1 day, 1 week, several weeks to a month or more) to enhance immunity when introduced to a subject to a broad range of types or serotypes of pathogenic organisms. For example, enhancing epitope availability or enhancing neutralization effects of a composition as a result of exposure to these elevated temperatures during storage. In certain embodiments, enhanced immunogenicity can occur simultaneously to the represented antigens of the complex or for enhance cross-reactivity. This aspect of the instant invention is surprising and unexpected as elevated temperatures typically are thought to have an adverse effect on immunogenicity of multi-complexed multi-component agents. In accordance with these embodiments, exposure to increased temperatures as reference above of a stabilized, lyophilized multi-targeted antigen (e.g. RG1 HPV VLP or other viral or bacterial complex such as alphavirus or flaviviruses), of the instant application, can increase cross-reactivity of the reconstituted complex against multiple pathogenic types or serotypes when introduced to a subject. In certain embodiments, a subject contemplated herein can be a human subject or other mammalian subject such as a pet or livestock exposed to or at risk of infection from a pathogen.

Certain embodiments disclosed herein can include kits of use for storage and transport of one or more HPV construct disclosed herein, one or more container and/or one or more lyophilized HPV construct or broad-spectrum multi-targeted antigen complex. In certain embodiments, kits contemplated herein can be kits able to withstand elevated temperatures and/or low temperatures, for use at temperature-ranges as disclosed herein (e.g. 4° C. to about 80° C.). In accordance with these embodiments, a kit can include a container having a lyophilized RG1-VLP construct in trehalose and ammonium acetate or similar agent as disclosed herein.

### Examples

This disclosure is further illustrated by the following non-limiting examples. All scientific and technical terms have the meanings as understood by one with ordinary skill in the art. The examples which follow illustrate the methods in which the chimeric compositions of the present disclosure may be prepared and used and are not to be construed as limiting the disclosure in sphere or scope. The methods may be adapted to variation in order to produce compositions embraced by this disclosure but not specifically disclosed. Further, variations of the methods to produce the same compositions in somewhat different fashion will be evident to one skilled in the art based on the present disclosure.

### Example 1

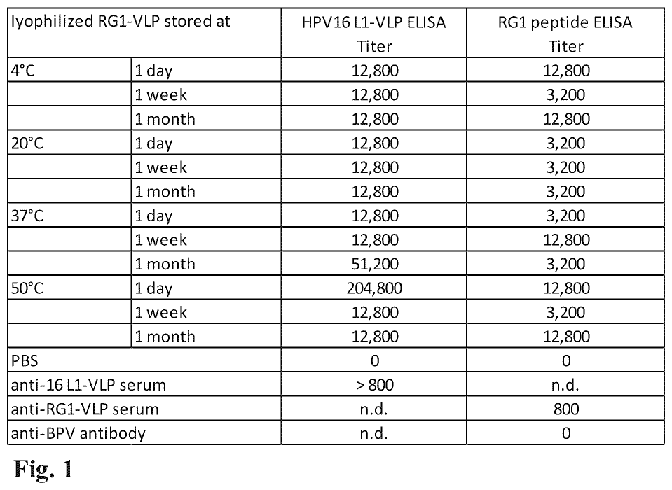

In certain exemplary methods, broad-spectrum HPV immunogenic compositions were tested in various formulations for stability at elevated temperatures. In one method, an ELISA assay was performed to assess titer of various formulations subjected to lyophilization and storage for prolonged stability. In exemplary FIG. 1, immune sera samples raised against lyophilized and reconstituted formulations of an exemplary construct, RG1-VLP that had been stored under various temperature conditions were tested by an HPV16 L1-VLP and RG1 peptide ELISA in 4-fold serial dilutions (1:200-1:204,800). Rabbit sera raised against HPV16 L1-VLP and RG1-VLP, and a BPV L1-raised monoclonal antibody, were used as positive or negative controls. Titers were graded positive for mean OD values greater than OD of pre-sera+3 standard deviations, n.d. indicate not determined. See FIG. 1 where stability is demonstrated at various temperatures up to an elevated temperature of about 50° C.

As illustrated in FIG. 1, titers were maintained at all temperatures tested.

### Example 2

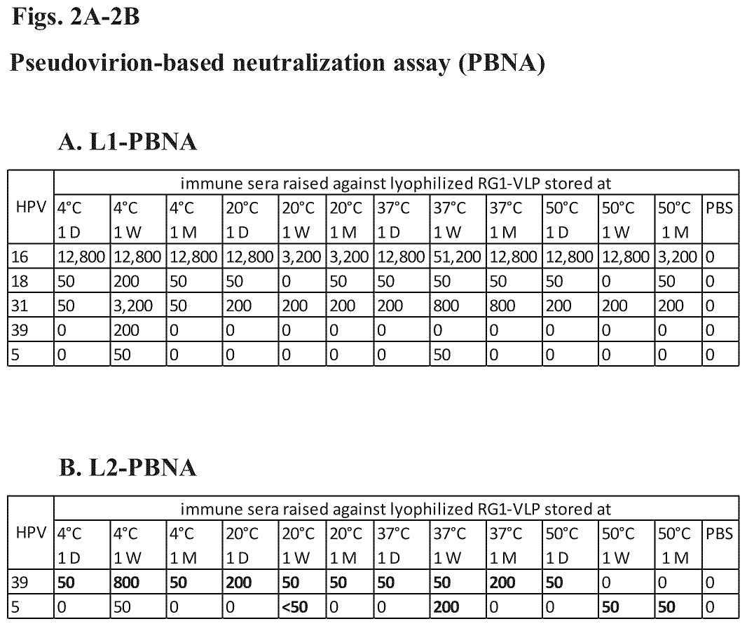

In another exemplary method, a pseudovirion-based neutralization assay (PBNA) was performed after storage of the HPV constructs at various temperatures and times. As illustrated in FIG. 2A and FIG. 2B L1-PBNA was performed to detect neutralizing antibodies against hr HPV16, and cross-neutralizing antibodies against hr HPV18, 31, 39 and cutaneous Beta type HPV5. L2-PBNA (See for example Day 2012) was performed against HPV39 and HPV5 to more sensitively detect potential cross-neutralization, and improved antibody titers detected were demonstrated (See the bold print). Surprisingly, cross-neutralizing titers against multiple HPV types such as HPV types 8, 18 31 and 39 were enhanced (instead of reduced) after a thermal treatment consisting of incubation for 1 month at 50° C.

### Example 3

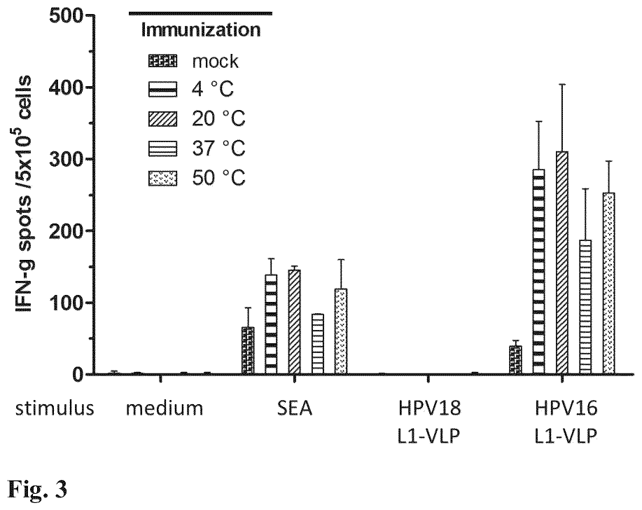

In another exemplary method, splenocytes were harvested from groups immunized with lyophilized RG1-VLP in certain exemplary compositions stored for 1 month at 4° C., 20° C., 37° C. or 50° C. and ex vivo stimulated with either HPV16 or HPV18 L1-VLP, or medium and Staphylococcus aureus enterotoxin A (SEA) as controls. Evaluation was performed using an ImmunoSpot® Analyzer (CTL) and Immunospot Software 5.0. (Scc for example, FIG. 3)

It was demonstrated in these exemplary methods that high-titer antibodies directed against HPV16 L1-VLP and the RG1 peptide were detected in all lyophilized RG1-VLP-raised immune sera by ELISA (FIG. 1). Notably, antibody titers were maintained even when lyophilized RG1-VLP were stored at elevated temperatures. Immune sera were neutralizing by L1-PBNA against HPV16 (titers of 3,200-51,200) and cross-neutralizing against hr HPV18, 31 and 39, and Beta HPVS (titers ranging from 50-3,200) in the majority of temperature groups (FIGS. 2A and 2B). Improved cross-neutralization was detected by more sensitive L2-PBNA particularly against HPV39 (titers of 50-800) and for some groups against HPVS (titers of <50-200). In this example, following incubation at higher temperatures lyophilized RG1-VLP maintain the ability to induce (cross-) neutralization with a trend towards reduced cross-neutralization seen in the highest temperature group (50° C.). By ELISPOT (FIG. 3), IFNy was induced by stimulation of splenocytes with HPV16 L1-VLP, but not HPV18 L1-VLP, in all tested storage temperature groups, which indicates maintained ability to raise a T cell response regardless of storage temperature of the RG1-VLP.

### Example 4

### Preparation of Lyophilized RG1 VLPs in Thermostable Glassy Matrices

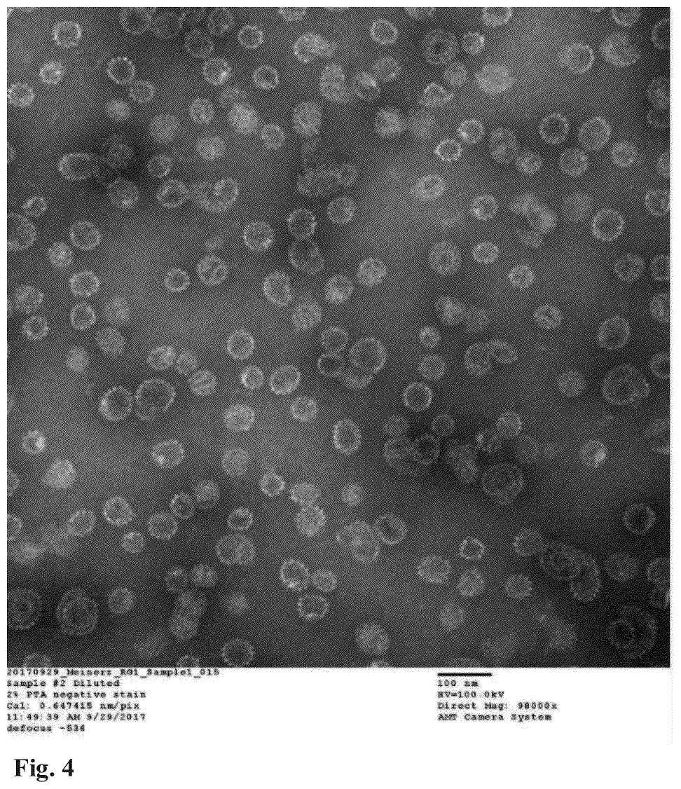

In one exemplary method, RG1-HPV VLPs were buffer-exchanged into a solution containing 100 mM histidine, pH7.1 Scanning electron micrographs (Sec FIG. 4) of the RG1-HPV VLP solutions revealed the presence of intact virus-like particles, with spiky protuberances. The solutions of RG1 HPV16 VLPs were mixed with trehalose and alum to form a mixture containing 10 wt/vol % trehalose, 0.5 mg/mL Alhydrogel® alum microparticles, and 0.1 mg/mL RG1 HPV VLPs. Other solutions were prepared in a similar fashion, but additionally contained 0.05 mg/mL of the immune co-stimulatory agent monophosphoryl lipid A. 1 mL aliquots of the solutions were filled into 3 mL Schott Fiolax lyophilization vials. The vials were placed on precooled (−40° C.) shelves of a Lyostar pilot-scale lyophilizer. Samples were dried under vacuum (60 mTorr) and vials were sealed under nitrogen. Samples of the lyophilized formulations were stored in temperature-controlled incubators at 4, 20, 37 and 50° C. for a period of 1 day, 1 week, and 1 month. After storage, samples were reconstituted with 1 mL of water for injection, and 100 microliter doses of the resulting solution were administered to mice. FIG. 4 represents an exemplary image of a scanning electron micrograph of RG1-HPV VLPs demonstrating intact virus-like particles after buffer exchange into 100 mM histidine, pH 7.1.

### Example 5

### Preparation of Lyophilized RG1 VLPs in Thermostable Glassy Matrices: Lyophilized RG1-VLP Immunizations

In one exemplary method, immunogenicity/thermostability of lyophilized in comparison to non-lyophilized RG1-VLP were incubated at increased temperatures for approximately one month. In these methods, RG1-VLP are virus-like particles (VLP) assembled from chimeric Human Papillomavirus (HPV) type 16 L1 major capsid protein incorporating the cross-neutralization epitope, RG1′ of HPV16 L2 (e.g. 20 amino-acid residues 17-36). RG1-VLPs were mixed with aluminum hydroxide (aluminum agent: ‘alum’) at an exemplary ratio of 1 μg RG1-VLP plus 5 μg alum. Other ratios than 1:5 are contemplated of use in the current disclosure such as 1:1; 1:2; 1:3; 1:4; 1:6; 1:7; 1:10; 1:15; 1;20 or the like. The broad-spectrum multi-antigen-containing formulation was lyophilized to generate a dry powder vaccine or, as control, was left untreated as liquid.

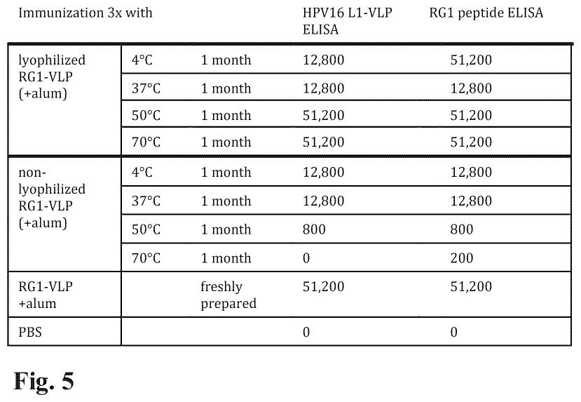

To evaluate thermostability of a broad-spectrum multi-antigen-containing complex (e.g, RG1-VLP) formulation to elevated temperatures, lyophilized and untreated alum-adjuvanted RG1-VLP, respectively, were incubated at about 4, 37, 50, or 70° C. over about a one-month period, transported at 4° C. overnight, followed by analysis of immunogenicity. See for example, FIG. 5.

### Example 6

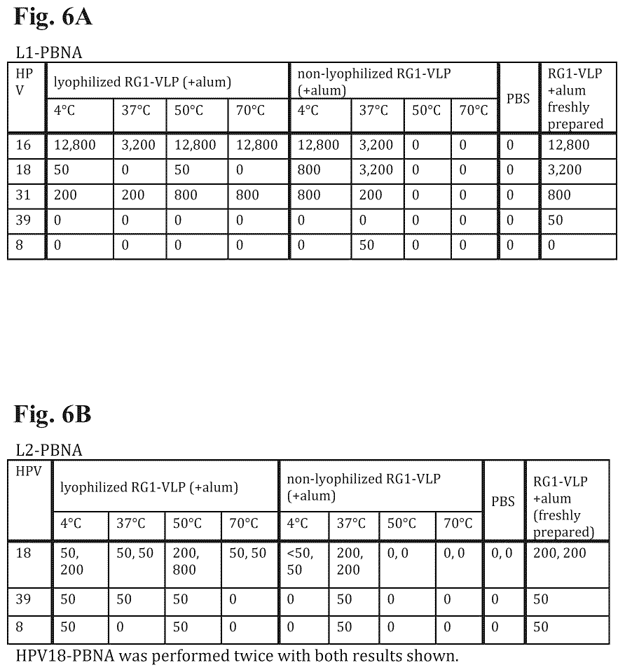

Following extended control and elevated temperature treatment, lyophilized broad-spectrum multi-antigen-containing complexes (e.g. RG1-VLP) were reconstituted in PBS, female Balb/c mice (groups of n=5) were immunized (2 μg VLP+10 μg alum per mouse and dose) 3 times intramuscularly (intramuscular: IM) in 2 weeks intervals (weeks 0, 2, 4). Immunizations of mice with non-lyophilized (liquid) treated broad-spectrum multi-antigen-containing complex (RG1-VLP)+alum, or untreated liquid broad-spectrum multi-antigen-containing complex (RG1-VLP)+alum (RG1+alum; RG1-VLP were treated with alum-adjuvant immediately before each immunization), or phosphate-buffered saline (PBS) alone served as a negative control. (Pre-) immune sera were drawn before immunization and two weeks after the final boost (week 6), pooled for groups (5 sera each) and analyzed by HPV16 L1-VLP and RG1 peptide ELISA. See for example, FIGS. 5A and 5B (Cross-) neutralizing activity against high-risk mucosal HPV16, 18, 31, 39, or high-risk cutaneous beta HPV5, 8 was assessed using L1- and L2-pseudovirion-based neutralization assays (PBNA). See for example, FIGS. 6A and 6B.

### Example 7

In another embodiment, antibody assays using an ELISA testing system demonstrated that using broad-spectrum multi-antigen-containing complex (RG1-VLP) (generated in Sf9 insect cells and purified by gradient centrifugation) as vaccine antigen, respectively, indicate the induction of prominent antibody titers (e.g. 12,800-51,200) against both HPV16 L1 and L2 antigen components in mice immunized with lyophilized vaccine irrespective of extended incubation at higher temperature (FIG. 1, 5). In contrast, liquid vaccine formulations incubated at 50° C. or 70° C. induced largely reduced (200-800) or undetectable (0) ELISA titers at these elevated temperatures (FIG. 5). Sera generated against freshly prepared RG1-VLP+alum formulation, or PBS served as positive and negative controls, respectively.

(Cross-) neutralization of immune sera against high-risk mucosal types HPV16/18/31/39 and cutaneous Beta HPV8 was further assessed by L1- and L2-pseudovirion-based neutralization assays (PBNA) (2, 3) Sera were serially diluted 4-fold from 1:50 to 1:204,800; pre-sera diluted 1:50 were non-neutralizing (not shown).

The L1-PBNA detected type-specific neutralization against HPV16 (titers of 3,200-12,800) in sera of mice treated with lyophilized vaccine formulation stored at 4, 37, 50, 70° C. (FIG. 6A). In contrast, storage of the (non-lyophilized) liquid immunogenic broad-spectrum multi-targeted antigens for one month at 50 or 70° C. destroyed immunogenicity, indicated by undetectable neutralizing activity to HPV 16 (titer of 0) by L1-PBNA.

### Example 8

When immune sera were tested for cross-neutralization against multiple antigens, HPV18/31/39/8, titers ranging from 0-3200 were observed for groups of mice vaccinated by both lyophilized or non-lyophilized (liquid) RG1-VLP stored for prolonged time at temperatures of 4, 37, 50, 70° C. Sera raised against freshly prepared RG1-VLP+alum formulation, or PBS served as positive and negative controls, respectively. See for example FIG. 6A.

Immune sera were further analyzed by L2-PBNA, which is more sensitive for detection of neutralizing antibodies directed to L2 (RG1). See for example FIG. 6B. Sera were serially diluted 4-fold from 1:50 to 1:12,800; pre-sera diluted 1:100 were non-neutralizing (not shown).

By L2-PBNA, sera from mice immunized with liquid broad-spectrum multi-targeted antigen (RG1-VLP)+alum incubated at 50° C. or 70° C. for 1 month showed undetectable neutralization to HPV18, 39, 8 (FIG. 6B). In contrast, lyophilized vaccine formulation exposed to the same thermal conditions induced neutralization titers of 50-800 following 50° C., and titers of 0-50 following 70° C. incubation to HPV18, 39, 8 types (FIG. 6B). Neutralization titers are shown. HPV18-PBNA was performed twice with both results shown.

It is noted that these observations indicate that lyophilization of a broad-spectrum multi-targeted antigen complex (e.g. RG1-VLP) formulated in alum confers thermostability to elevated temperature exposure over an extended period of one month with respect to immunogenicity, e.g., the ability to induce antisera that (cross-) neutralize several high-risk mucosal and cutaneous pathogens (e.g. HPV types). However, a trend towards reduced cross-neutralization was seen in the highest temperature treatment group (70° C.). Therefore, incubation between 4° and 60° C. may be preferred for certain complexes contemplated herein.

### Example 9

To determine if immunization with lyophilized RG1-VLP exposed to elevated temperatures induces a cellular immune response, spleens of two mice per group immunized with liquid or lyophilized broad-spectrum multi-targeted antigen (e.g. RG1-VLP)+alum stored at 4, 50, or 70° C. for one month were removed, splenocytes were harvested and pooled, and stimulated with 1 μg HPV16 or HPV18 L1 VLP; or 10 μg Staph. Aureus Enterotoxin (SEA), or medium alone as controls.

### Example 10

In other exemplary methods, a T-cell response, which can be indicated by measuring IFN-γ levels using for example, ELISPOT, was induced in splenocytes from mice immunized with lyophilized and non-lyophilized broad-spectrum multi-targeted antigen complex formulations (e.g. RG1 VLP) independent of the extended incubation temperature (See for example, FIG. 3, 7). Compositions and methods for providing superior multi-targeted antigen complex formulations having improved immunogenicity have been identified while reducing the need for cold-chain transport requirements, facilitating global distribution of these broad-spectrum multi-targeted antigen complexes for administration to humans, livestock or other animals.

### Materials and Methods

In certain methods, aluminum hydroxide (alum)-adjuvanted RG1-VLP were lyophilized by lyophilizing with trehalose. Aliquots of a dry-powder formulation were incubated at 4° C., 20° C., 37° C. or 50° C. for either 1 day, 1 week or 1 month, resuspended and used to immunize groups of Balb/c (n=5) in a 3-dose regime (211 g VLP/dose; week 0/2/4; blood finally drawn at week 6). Immune sera were pooled for groups and tested by HPV16 L1-VLP and RG1-peptide ELISA, as well as L1- and L2-based pseudovirion neutralization assays (L1- and L2-PBNA). Further, a T cell response was evaluated by IFNy ELISPOT using splenocytes that were pooled for groups.

All of the COMPOSITIONS and METHODS disclosed and claimed herein can be made and executed without undue experimentation in light of the present disclosure. While the compositions and methods have been described in terms of particular embodiments, it is apparent to those of skill in the art that variations maybe applied to the COMPOSITIONS and METHODS and in the steps or in the sequence of steps of the methods described herein without departing from the concept, spirit and scope herein. More specifically, certain agents that are both chemically and physiologically related may be substituted for the agents described herein while the same or similar results would be achieved. All such similar substitutes and modifications apparent to those skilled in the art are deemed to be within the spirit, scope and concept as defined by the appended claims.

What is claimed is:

1-19. (canceled)

20. An immunogenic composition comprising:
a pre-formed broad-spectrum multi-targeted human papilloma virus (HPV) antigen construct wherein the broad-spectrum multi-targeted HPV antigen construct comprises a VLP and wherein the VLP comprises a VLP from HPV L1 VLPs having at least one HPV L2 epitope insertion;
one or more non-reducing disaccharide agents; and
one or more volatile salts;
wherein the immunogenic composition is essentially dried, and the essentially dried composition has been incubated for a pre-determined period of at elevated temperatures of 40° C. to about 70° C. for at least one day to enhance immunogenicity of the immunogenic composition.

21. The immunogenic composition according to claim 20, wherein the one or more non-reducing disaccharide is selected from the group consisting of trehalose, sucrose, lactose, or combinations thereof.

22. The immunogenic composition according to claim 20, wherein the one or more volatile salts comprise one or more of ammonium acetate, ammonium formate, ammonium carbonate, ammonium bicarbonate, triethylammonium acetate, triethylammonium formate, triethylammonium carbonate, trimethylamine acetate trimethylamine formate, trimethylamine carbonate, pyridinal acetate, pyridinal formate, or combinations thereof.

23. The immunogenic composition according to claim 20, further comprising an aluminum salt adjuvant.

24. The immunogenic composition according to claim 20, wherein the broad-spectrum multi-targeted HPV antigen construct comprises a VLP assembled from HPV L1 VLPs having an HPV L2 epitope insertion into a surface loop of at least one L1 protein.

25. The immunogenic composition according to claim 20, wherein the broad-spectrum multi-targeted HPV antigen construct comprises RG1 HPV16 VLP.

26. The immunogenic composition according to claim 20, wherein the broad-spectrum multi-targeted HPV antigen construct comprises two or more different HPV pathogens or serotypes comprising at least two assembled subunits of the two or more different HPV pathogens or serotypes.

27. An immunogenic pharmaceutical composition comprising, a construct composition according to claim 20, and a pharmaceutically acceptable excipient.

28. The pharmaceutical composition according to claim 27, of use as a vaccine for administering to a subject to reduce onset of a health condition related to two or more HPV serotypes.

29. A method of preparing an immunogenic composition, the method comprising:
(a) combining a pre-formed broad-spectrum multi-targeted human papilloma virus (HPV) antigen construct wherein the broad-spectrum multi-targeted HPV antigen construct comprises a VLP and wherein the VLP comprises a VLP from HPV L1 VLPs having an HPV L2 epitope insertion, with one or more non-reducing disaccharide agents; and
one or more volatile salts in a buffer making a liquid immunogenic composition;
(b) freezing the liquid immunogenic composition;
(c) lyophilizing the frozen immunogenic composition creating an essentially dry formulation of the immunogenic composition;
(d) exposing the essentially dry formulation of the immunogenic composition to elevated temperatures of 40° C. to about 70° C. for at least one day prior to delivery of the immunogenic composition to a subject; and
(e) enhancing immunogenicity of the immunogenic composition.

30. The method according to claim 29, wherein the one or more non-reducing disaccharide is selected from the group consisting of trehalose, sucrose, lactose, or combinations thereof.

31. The method according to claim 29, wherein the one or more volatile salts comprise one or more of ammonium acetate, ammonium formate, ammonium carbonate, ammonium bicarbonate, triethylammonium acetate, triethylammonium formate, triethylammonium carbonate, trimethylamine acetate trimethylamine formate, trimethylamine carbonate, pyridinal acetate, pyridinal formate, or combinations thereof.

32. The method according to claim 29, wherein exposing the essentially dry formulation of the immunogenic composition to elevated temperatures comprises, exposing the lyophilized immunogenic composition to temperatures of 40° C. to about 70° C. for at least one week to several months prior to delivery of the immunogenic composition to the subject.

33. The method according to claim 29, wherein the broad-spectrum multi-targeted HPV antigen construct comprises a VLP assembled from HPV L1 VLPs having an HPV L2 epitope insertion into a surface loop of at least one L1 protein.

34. The method according to claim 29, wherein the freezing step comprises one of tray freezing, flash freezing, shelf freezing, spray-freezing, or shell-freezing.

35. A method for eliciting an enhanced immune response to one or more HPV types in a subject, the method comprising administering to the subject a reconstituted immunogenic composition according to claim 27 and eliciting an immune response to two or more HPV types in the subject.

36. A method for enhancing cross-reactivity in a subject of immune responses to an RG1-HPV immunogenic composition, the method comprising lyophilizing an aluminum salt adjuvanted RG1-HPV antigen and further exposing the lyophilized aluminum salt adjuvanted RG1-HPV antigen to elevated temperatures of about 40° to about 70° C. for at least one week to form an improved aluminum salt adjuvanted RG1-HPV antigen and administering the improved aluminum salt adjuvanted RG1-HPV antigen to the subject to elicit an enhanced immune response.

37. The method according to claim 36, wherein the RG1-HPV antigen comprises an RG1 HPV16 VLP antigen.

38. A kit comprising an immunogenic composition according to claim 20; and at least one container.
